(19) United States
(12) Patent Application Publication
(10) Pub. No.: US 2010/0168726 A1
(43) Pub. Date: Jul. 1, 2010

(54) Cryogenic Dispensing System and Method for Treatment of Dermatological Conditions

(76) Inventor: Marc Arthur Brookman, Livonia, MI (US)

Correspondence Address: MARC A. BROOKMAN, 19538 WESTMORE AVE., LIVANIA, MI, 48152, US

(21) Appl. No.: 12/347,715

(22) Filed: Dec. 31, 2008

(51) Int. Cl. A61B 18/02

(52) U.S. Cl. 606/22

(57) ABSTRACT

A cryogenic dispensing system has a cartridge containing a cryogenic material of limited dose and a housing to hold and dispense the cryogenic material at a controlled flow rate. The housing has a hollow interior and has an inlet opening adapted to receive a cartridge containing a cryogenic material and an outlet opening adapted to deliver cryogenic material from the interior to a wart, other tissue to be treated or the like. The hollow interior of the housing has an intermediate portion adapted to receive the insert member which has an engineered flow path therethrough and a method of starting the flow, where when opened, places the inlet in fluid communication through the flow path with the outlet. Preferably, the system includes a cartridge which contains liquid CO₂. A masking ring may also be provided downstream of the flow path for applications of CO₂ to selected dermatological areas such as a wart or the like.

### FIELD OF THE INVENTION

The present invention relates to a cryogenic dispensing system. More particularly, the present invention relates to a cryogenic dispensing system which has a cartridge of liquid cryogenic material of limited dose, preferably liquid carbon dioxide and use of the system for treatment of dermatological conditions such as common and plantar warts.

### BACKGROUND OF THE INVENTION

Licensed medical doctors have used cryogenic methods for the treatment of dermatological conditions for many years. For example, a common treatment for warts has been to freeze the tissue by applying liquid nitrogen onto them. However, liquid nitrogen is not available as an “over the counter” material. Because of its reactivity, its use is best reserved for professionals.

There are commercially available compositions for freezing warts which do not employ liquid nitrogen and which are available “over-the-counter.” For example, “Freeze Off” which is available from Compound W involves the use of a swab-like applicator to apply a composition of dimethyl ether, propane, and isobutane. Also, Wartner's “Wart Removal System” comprises a dimethyl ether, and propane composition which is sprayed into a foam pad which is then applied to the wart.

Thus, there are cryogenic systems presently available “over-the-counter” for the treating of warts; however, it is believed that there remains a need for an improved system for application of a cryogenic treatment agent for treatment of dermatological conditions such as common or plantar warts. There also remains a need for an improved system for cryogenically treating dermatological conditions which is suitable for “over the counter” sale to and use by ordinary consumers. It, of course, would be desirable to have a system which included a cryogenic device but which employed relatively inexpensive and readily available cartridges containing liquid carbon dioxide which can be easily stored and used. These and other advantages of the present invention will be apparent from the following specification taken in conjunction with the claims and drawings appended hereto.

### BRIEF DESCRIPTION OF THE FIGURES

FIG. 1 is an isometric view of a preferred embodiment of a cryogenic dispensing system of the present invention;

FIG. 2 is a front view of said cryogenic dispensing system.

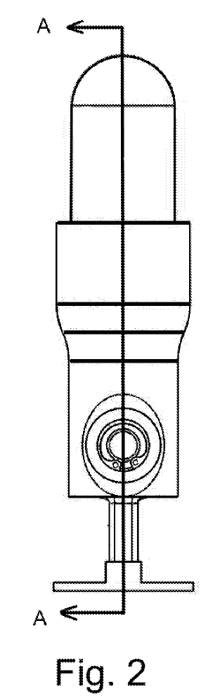

FIG. 3 is a sectional view of the cryogenic dispensing system along line A-A of FIG. 2.

FIG. 4 is an enlarged sectional view of the central portion of the dispensing system of FIG. 3

### SUMMARY OF THE INVENTION

A cryogenic dispensing system includes a cartridge of cryogenic material, preferably liquid carbon dioxide, and a cryogenic dispensing device. The cryogenic dispensing device has a hollow interior and has an inlet opening adapted to receive the cartridge containing a cryogenic material, a valve mechanism to start the flow of the cryogenic material, and an outlet opening adapted to deliver cryogenic material from the interior of the cartridge to a dermatological condition such as a wart or other tissue to be treated or the like. Preferably, the cartridge is sized to contain a single dose of liquid cryogenic material and the outlet opening is engineered to control the flow rate of the cryogenic material. A masking tip may also be provided downstream of the flow path for precise application of cryogenic material to a selected dermal area.

### DESCRIPTION OF THE INVENTION

In a preferred embodiment of the present invention, a cryogenic system is provided for applying a cryogenic material onto a surface for the treatment thereof. Preferably, the surface is a dermal surface and the treatment is of a skin condition such as a common or plantar wart. Also, preferably, the system comprises a device which in combination with a selectively sized cartridge provides a measured amount of cryogenic material for a single treatment at a metered flow rate. The present invention also relates to a method of using the system to treat a dermatological condition.

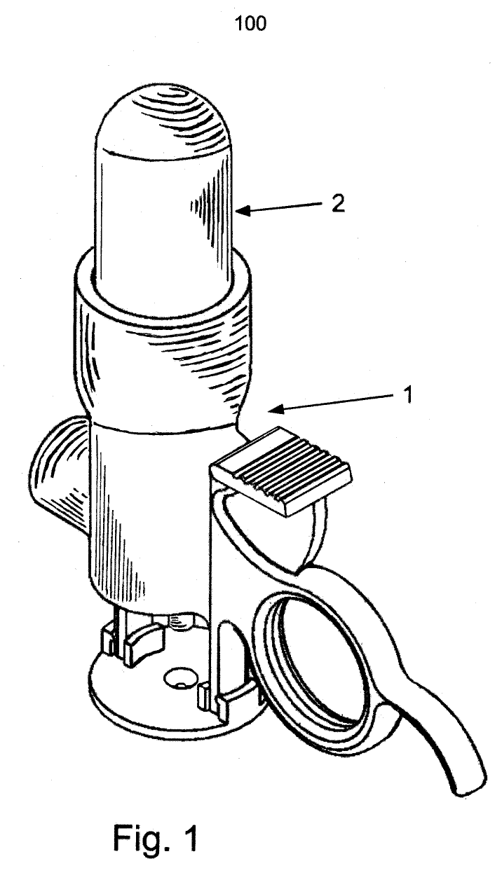

Referring to FIG. 1, a preferred embodiment of a cryogenic dispensing system of the present invention is shown and indicated generally by the numeral 100. The cryogenic dispensing system 100 comprises of a dispensing device 1 and cartridge 2 which contains a cryogenic material such as a liquid/gas mixture of carbon dioxide (CO₂). Preferably, cartridge 2 is a conventional CO₂ cartridge such as are readily commercially available. Also preferably, cartridge 2 is of a size to contain an amount of cryogenic material for a single treatment.

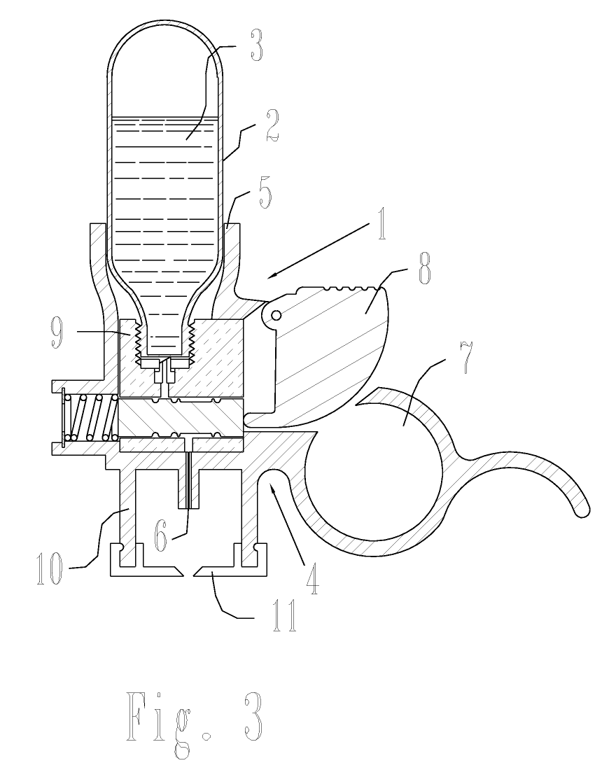

In FIG. 3, dispensing device 1 is intended to be held by the hand of one manipulating the device and has a housing 4 which could be made of a polymeric or metallic material or the like. The upper part of housing 4 defines a cylinder inlet opening 5 which receives and supports cartridge 2 which contains the cryogenic material 3. The lower part of housing 4 has an outlet opening 6 and shield support legs 10 which are used to hold the shield insert 11. Housing 4 also has finger loop handle 7 to facilitate holding and manipulation of dispensing device 1 and carries a trigger 8 for activation of the dispensing device as set forth in more detail below.

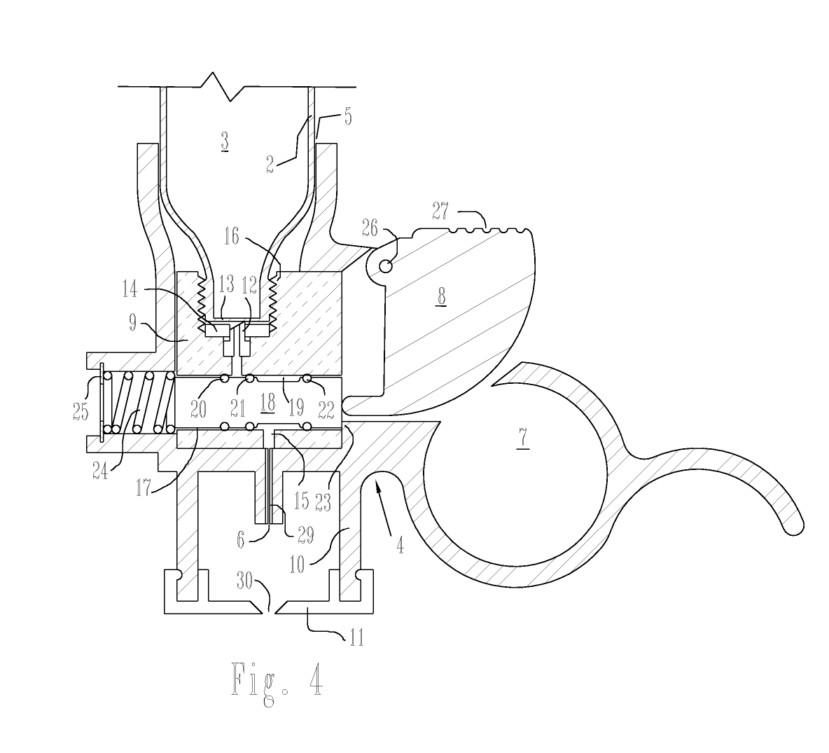

Referring to FIG. 4, insert 9 carries a hollow lance 12 which can be machined therewith or press fit therein and which is adapted to pierce the membrane 13 of cartridge 2. Seal 14 creates a seal between cartridge 2 and insert 9 prior to lance 12 piercing the membrane 13 of cartridge 2 and maintains a seal until the cryogen has been discharged. Insert 9 also has a lower passageway 15 at the lower end thereof which lines up with outlet opening 6 of housing 4. Insert 9 is shown in the Figures with a threaded bore 16 for threadably receiving cartridge 2 although it will be appreciated that cartridge 2 may be press fit or otherwise secured in place by other means if desired.

If the housing 4 is made of a polymeric material it would be desirable to have a metallic insert 9 press fit, insert molded, or otherwise fixedly secured as by adhesive or the like into said housing 4 to allow strength for threadibly attaching cartridge 2. If the housing 4 is made of a metallic material, said housing 4 and insert 9 could be combine into one piece.

Insert 9 further has a transverse cylindrical bore 17 into which valve spool 18 is positioned. Valve spool 18 has a cylindrical shape closely but slidably fitting within bore 17 and has an annular groove 19 extending completely around its circumference with elastomeric seals 20, 21, and 22 also extending therearound. Valve spool 18 is biased rightwardly against shoulder 23 of housing 4 by spring 24 in bore 17, spring 24 being compressed between valve spool 18 and snap ring 25. Trigger 8 is pivotally attached to housing 4 by pivot pin 26 and has a shelf 27 which can be manipulated to pivot trigger 8 to urge valve spool 18 leftwardly against spring 24 to thereby align annular groove 19 with the lower open end of lance 12 and lower passageway 15. The cryogenic material enters outlet opening 6 from lower passageway 15. Outlet opening 6 is either drilled to the proper size to create the engineered flow path 29 or has a piece of tubing insert molded, press fit, or adhesively affixed therein to control the flow of cryogenic material. By changing the length and inside diameter of engineered flow path 29, the cryogenic material can be economically controlled to a rate suitable for the cryogenic treatment of dermatological conditions. Different cryogenic fluids have different kinematic properties and thus may require different length and diameter engineered flow paths. Likewise different dermatological conditions may require different flow rates of a similar cryogenic material and thus may require different length and inside diameter engineered flow paths. It should be obvious to those skilled in the art that the required flow rates and corresponding length and inside diameter of the engineered flow path for various conditions and cryogenic materials can be determined quite easily through analytical and experimental methods. Therefore it should also be obvious that the design shown in the preferred embodiment of a cryogenic dispensing system of the present invention can be economically made to be intrinsically safe by controlling the flow rate and limiting the dose.

The design of the present invention facilitates the practical and economical manufacture thereof. Housing 4, trigger 8, and valve spool 18 can be readily molded of polymeric material in any conventional fashion, it being kept in mind that housing 4 and valve spool 18 must be tolerant of cryogenic temperatures. In assembly of cryogenic dispensing device 1, insert 9 is secured within housing 4. Valve spool 18 with seals 20, 21 and 22 in place is put into insert 9 through housing 4 by extending it through bore 17 followed by spring 24 and snap ring 25. Trigger 8 can be attached to housing 4 by means of pin 26 at any time during the assembly. It should be noted that the housing 4 and the insert 9 could be molded as one piece from plastic or die cast or machined from metal. In either case it may require a hardened insert for lance 12 to be capable of piercing the membrane 13 of cylinder 2. It is conceivable that engineered flow path 29 could be integral to housing 4 by way of molding or drilling, however holes of the required diameter range for most cryogenic fluids, including CO₂ are not easily injection molded or drilled by conventional means and thus it is likely more practical to insert and secure a tube after the housing 4 has been molded or machined.

In operation of the preferred embodiment of a cryogenic dispensing system 100, one first attaches shielding insert 11, with the proper sized masked opening 30 for the condition to be treated, on shield support legs 10 of housing 4, a cryogenic cartridge 2 is inserted through cylinder inlet opening 5 into insert 9 in housing 4 of dispensing device 1 by threading the cartridge into tapped bore 16 of insert 9. Masked opening 30 of shielding insert 11 is placed over the dermal condition to be treated. Trigger 8 is manipulated to move valve spool 18 leftwardly which opens the flow path through lance 12, annular groove 19 of valve spool 18, lower passageway 15, engineered flow path 29, and outlet opening 6. The cryogenic material exits outlet opening 6 and is applied to the dermal condition that is exposed by masked opening 30 of shielding insert 11 for a fixed amount of time at the required flow rate. Thus, cryogenic delivery system 100 is well suited to safely freeze dermatological areas for treatment. Because the present invention limits the amount of cryogenic material that can be applied, the design in intrinsically safe and suited but not limited to “over the counter” use.

Clearly, minor changes may be made in the shape and construction of the invention described without departing from the material spirit thereof. It is desired that the invention shown and described herein not be limited to its exact form, but allowed to include all such as properly come within the scope claimed.

What is claimed is:

1. A cryogenic dispensing system:
a dispensing devise;
a cartridge containing a fixed amount of cryogenic material for a single dose
said dispensing devise adapted to receive said cartridge containing a cryogenic material, a housing and an intermediate interior portion adapted to connect said cartridge and an outlet opening.
said intermediate interior portion containing a fixed engineered flow path capable of controlling the flow of said cryogenic material in said cartridge and said outlet opening, and a method of starting the flow of said cryogenic material.

2. The cryogenic dispensing device of claim 1, wherein said intermediate interior portion has a coupling element for receiving and holding said cartridge and a lance with a bore for penetrating into said cartridge to place the interior thereof in fluid communication with said engineered flow path.

3. The cryogenic dispensing device of claim 1, wherein said intermediate interior portion contains a valve, to start the flow of said cryogenic material to said engineered flow path and outlet opening.

4. The cryogenic dispensing device of claim 1, wherein said housing has extended legs to space the exit end of the engineered flow path from the dermal surface.

5. The cryogenic dispensing device of claim 4, wherein said extended legs are configured to accept masking inserts to create various sized masked areas to be treated.

6. The cryogenic system of claim 1, wherein said engineered flow path is sized to deliver liquid carbon dioxide to said outlet opening in an amount from about 0.2 to about 3.5 grams per second.

7. A method of treating dermatological conditions such as a wart or the like, comprising the steps of:
providing a cryogenic dispersing system having:
a cartridge comprising liquid cryogenic material of limited dose; and
a dispensing device having a housing;
said housing containing an intermediate interior portion adapted to receive said cartridge,
said intermediate interior portion defining an engineered flow path between said cryogenic material in said cartridge and outlet opening, and a valve capable of starting the flow of cryogenic material;
said housing containing extended legs for spacing of the exit end of the engineered flow path from the dermal surface to be treated.
said extended legs containing a method of mounting a masking ring;
placing said masking ring in contact with the dermal surface to locate the outlet opening proximate to said dermatological condition; and
allowing said liquid carbon dioxide to flow through said engineered flow path to said outlet opening and onto said dermatological condition.
